(19) United States
(12) Plant Patent Application Publication
(10) Pub. No.: US 2016/0360674 P1
(43) Pub. Date: Dec. 8, 2016

(54) Oncidium orchid plant named 'ONCDBALCOJ'

(50) ;

(71) Applicant: ANTHURA B.V., Bleiswijk (NL)

(72) Inventor: Gert-Jan Hoogendoorn, Woubrugge (NL)

(21) Appl. No.: 14/545,660

(22) Filed: Jun. 3, 2015

(51) Int. Cl. A01H 5/00

(52) U.S. Cl. PLT/311

(57) ABSTRACT

A new and distinct variety of Oncidium plant named ‘ONCDBALCOJ’, particularly characterized by having yellow flowers with a light brown spot in the area of the callus and striations on the sepals and petals, 1-3 peduncles, a peduncle that is short and moderate, leaves that are lanceolate and moderately elongated, and is propagated by tissue culture is disclosed.

### GENUS AND SPECIES

Oncidium altissimum Sw.

### VARIETY DENOMINATION

‘ONCDBALCOJ’

### BACKGROUND OF THE NEW PLANT

The present invention comprises a new and distinct cultivar of Oncidium plant, botanically known as Oncidium altissimum Sw. of the Orchidaceae family, and hereinafter referred to by the cultivar name ‘ONCDBALCOJ’.

Oncidium comprises a genus of about 330 species of herbaceous perennials many of which, or the hybrids thereof, are suitable for cultivation at the home or greenhouse. Oncidium thrives growing as lithophytes or terrestrials and is distributed throughout the American tropics and extending into South-Central America. All Oncidium exhibit a sympodial growth habit. The species typically have 4 leaves per mature, pseudobulb: 2 lanceolate leaves with acute apex grown on the apex of the pseudobulb and 2 leaves grown from the axis at the base (sympodial growth). The peduncles vary in size from 20 to over 60 cm.

Oncidium orchids in the horticultural trade are frequently used to furnish cut flowers for the florist trade or sold as flowering potted plants for home or interiorscape.

Oncidium produces upright or pendent lateral racemes to panicles, often with many showy flowers which open in succession beginning with the lowermost. The flowers possess three sepals and two petals; the lateral ones being alike. The lowermost petals, called the labellum, are three-lobed and are often more brightly-colored than the other flower segments. Flower colors include various shades of pink, white, yellow, and red-brown.

Oncidium orchids are typically propagated from seeds. Asexual propagation of Oncidium is often done from off-shoots which arise from the lower bracts of the inflorescence. The resulting plants are detached from the mother plants and may be planted in a suitable substrate.

The new Oncidium ‘ONCDBALCOJ’ is particularly characterized by its attractive and unique, yellow flowers with a light brown spot in the area of the callus and striations on the sepals and petals, a short and compact plant habit, economical propagation by tissue culture, rapid growth, and a plant dimension suitable for packaging and shipping to the market.

‘ONCDBALCOJ’ is a product of a planned breeding program conducted by the inventor Gert-Jan.Hoogendoorn, in Bleiswijk, The Netherlands.

The new Oncidium ‘ONCDBALCOJ’ originated from a cross made by the inventor in August 2008 in Bleiswijk, The Netherlands. The female parent is a yellow Oncidium pot plant named ‘60000-0041’ (unpatented) and the male parent is a yellow Oncidium pot plant named ‘60000-0522’ (unpatented). A single plant was selected in August 2011 and has been asexually reproduced repeatedly by meristem tissue culture in Bleiswijk, The Netherlands over a 2.5 year period. The new variety has been found to retain its distinctive characteristics through successive asexual propagations.

Asexual reproduction of ‘ONCDBALCOJ’ by tissue culture was first performed in September 2012 in Bleiswijk, The Netherlands and has demonstrated that the new cultivar is firmly fixed and retained through successive generations of asexual reproduction.

Plant Breeder's Rights for this variety have been applied for in Europe on Oct. 27, 2014. ‘ONCDBALCOJ’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Bleiswijk, The Netherlands.
1) Yellow flowers with light brown spot in area of callus and striations on the sepals and petals;
2) 1-3 peduncles;
3) Peduncle is short and moderate;
4) The shape of the leaf is lanceolate and moderately elongated; and
5) Plants are propagated by meristem tissue culture.

### DESCRIPTION OF THE PHOTOGRAPHS

This new Oncidium plant is illustrated by the accompanying photographs which show the overall plant habit including blooms and foliage of the plant; the colors shown are as true as can be reasonably obtained by conventional photographic procedures. The photographs are of a 40-week old plant grown in a greenhouse in Bleiswijk, The Netherlands in December 2014.

FIG. 1 shows the overall plant habit, including blooms and foliage of ‘ONCDBALCOJ’.

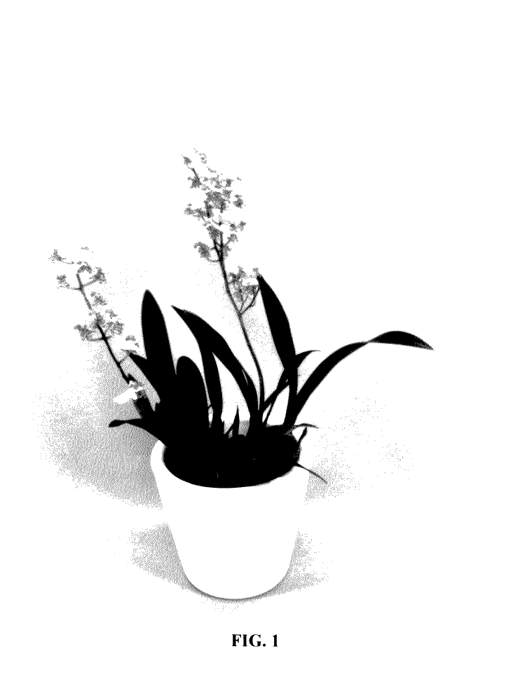

FIG. 2 shows a close-up of a flower of ‘ONCDBALCOJ’.

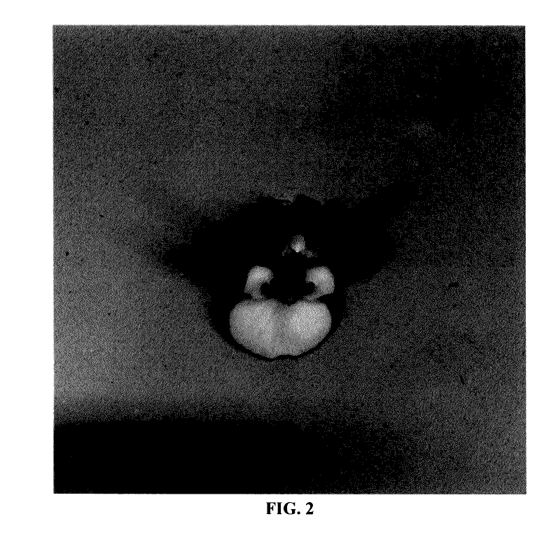

FIG. 3 shows a close-up of the leaves of ‘ONCDBALCOJ’.

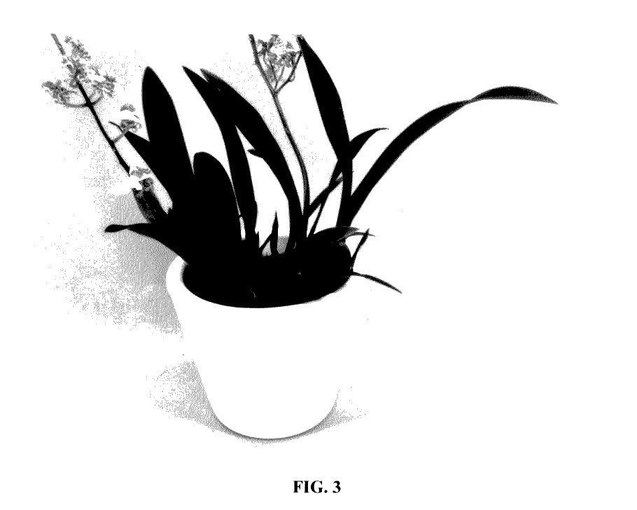

### DESCRIPTION OF THE NEW VARIETY

The following detailed description sets forth the distinctive characteristics of ‘ONCDBALCOJ’. The data which define these characteristics were collected from asexual reproductions carried out in Bleiswijk, The Netherlands. The plant history was taken on 40-week old plants which were planted from tissue culture in 9 centimeter pots and grown in a greenhouse between 20° C. to 25° C. for 40 weeks. Observations were made in December 2014. Color readings were taken under 4-6000 lux natural light in the greenhouse. Color references are primarily to The R.H.S. Colour Chart of The Royal Horticultural Society of London (R.H.S.) (2001).

### DETAILED BOTANICAL DESCRIPTION

Classification:
   Family.—Orchidaceae.
   Botanical.—Oncidium altissimum Sw.
   Common name.—Oncidium.
   Variety name.—‘ONCDBALCOJ’.
Parentage:
   Female parent.—Oncidium cultivar ‘60000-0041’ (unpatented).
   Male parent.—Oncidium cultivar ‘60000-0522’ (unpatented).
Propagation:
   Type.—Tissue culture.
Plant:
   Crop time (time to produce a finished flowering plant).—20 to 40 weeks for a 9 cm pot
   Growth habit of peduncle.—Standard, green leaves, raceme to panicle.
   Height (including pot, including inflorescence).—25.0 cm to 35.0 cm
   Vigor.—Moderate.
Roots:
   Root description.—White colored roots with white, transparent growing tips (The exact shades of white may vary with minimal changes of environmental conditions).
Pseudobulb:
   Shape.—Laterally compressed ovoid.
   Height.—3.7 cm to 4.2 cm.
   Length.—2.8 cm to 3.3 cm.
   Width.—2.0 cm to 2.3 cm.
   Color.—Green (RHS 146A).
Leaves:
   Mature leaves.—Quantity per plant: 2 to 4 leaves are produced before flowering Length (fully expanded): 8.5 cm to 9.5 cm Width: 1.7 cm to 2.3 cm Shape: Lanceolate Base shape: Moderately elongated Apex: Acute Leaf margin: Entire Color: Upper surface: RHS 146A Lower surface: RHS 146B Texture: Slightly rough Thickness: 0.70 mm to 0.80 mm Venation: Pattern: Parallel Color of the midvein: Upper surface: RHS 146A Lower surface: RHS 146B.
Peduncle:
   Quantity per plant.—1 to 3.
   Number of flowers per peduncle.—15 to 45 (number of flowers depends on the branching of the inflorescence).
   Length.—18.0 cm to 23.0 cm.
   Diameter.—2.0 mm to 3.0 mm.
   Strength.—Moderate.
   Aspect.—Upright.
   Texture.—Smooth.
   Color.—Green (RHS 146B).
   Internode length.—12.0 mm to 22.0 mm.
   Number of branches.—0 to 4.
   Callosities.—None.
Inflorescence description:
   Appearance.—Upright, raceme to panicle inflorescence with bilaterally symmetrical flowers that open in succession beginning with the lowermost flower.
   Inflorescence size.—Height (from base to tip): 100.0 mm to 130.0 mm Diameter: 1.4 mm to 1.9 mm.
   Flowering time.—All year; first flowers can be expected 5 to 9 months after planting in a 9 cm pot.
   Flower.—Height: 19.0 mm to 24.0 mm Diameter: 11.0 mm to 16.0 mm.
   Flower longevity.—On the plant: 5 to 10 weeks.
   Fragrance.—Present (Slight fragrance).
   Petals.—Shape: Ovate Length (from base to tip): 6.0 mm to 9.0 mm Width: 2.0 mm to 5.0 mm Color (when fully opened): Main color: Yellow (RHS 6C), striped greyed-orange (RHS 172A) At the base: Yellow (RHS 6C).
   Dorsal sepal.—Shape: Elliptic Length (from base to tip): 5.0 mm to 8.0 mm Width: 2.0 mm to 5.0 mm Color (when fully opened): Main color: Yellow (RHS 6C), striped greyed-orange (RHS 172A) At the base: Yellow (RHS 6C).
   Lateral sepals.—Shape: Ovate Length (from base to tip): 6.0 mm to 9.0 mm Width: 2.0 mm to 5.0 mm Color (when fully opened): Main color: Yellow (RHS 6C), striped greyed-orange (RHS 172A) At the base: Yellow (RHS 6C).
   Labellum (lip).—Length: 8.0 mm to 13.0 mm Width: 14.0 mm to 19.0 mm Color: Yellow (RHS 7C), greyed-orange towards the callus (RHS 167A).
   Callus.—Color: Greyed-orange (RHS 167A).
Reproductive organs:
   Arrangement.—The stamens, style and stigmas are fused into a single, short structure called the column, possessing one terminal anther with pollen grains united into pollinia, which are covered by an anther cap. The stigma is located under the column behind the pollinia. The ovary is inferior with three carpels present.
   Column.—Length: 3.0 mm to 4 0 mm Diameter: 2.0 mm Color: Green/yellow (RHS 143C, 6C).
   Pollinia.—Quantity: 2 Diameter: 0.2 mm to 0.3 mm Color: Yellow-orange (RHS 22A).
   Ovary.—Length: Not visible.
   Pedicel.—Length: 16.0 mm to 18.0 mm Diameter: 0.9 mm to 1.1 mm.
Disease, pest, and stress resistance: No specific resistance or susceptibility observed
Temperature tolerance: Not observed

### COMPARISON WITH PARENTAL AND COMMERCIAL VARIETIES

‘ONCDBALCOJ’ differs from female parent ‘60000-0041’ (unpatented) in that ‘ONCDBALCOJ’ has an acute leaf apex, whereas ‘60000-0041’ has an unequal obtuse leaf apex. Additionally, ‘ONCDBALCOJ’ has a shorter plant with smaller flowers than ‘60000-0041’.

‘ONCDBALCOJ’ differs from male parent ‘60000-0522’ (unpatented) in that ‘ONCDBALCOJ’ has an acute leaf apex and a smaller callus that is light brown, whereas ‘60000-0522’ has an unequal obtuse leaf apex and a larger callus that is darker brown. Additionally, ‘ONCDBALCOJ’ has a larger flower than ‘60000-0165’.

‘ONCDBALCOJ’ differs from commercial variety ‘SWEET SUGAR’ (unpatented) in that ‘ONCDBALCOJ’ has a smaller lip, a smaller pseudobulb and smaller pollinia than ‘SWEET SUGAR’.

I claim:

1. A new and distinct variety of Oncidium plant named ‘ONCDBALCOJ’, substantially as described and illustrated herein.
